(19) United States
(12) Patent Application Publication
(10) Pub. No.: US 2015/0065992 A1
(43) Pub. Date: Mar. 5, 2015

(54) SYRINGE WITH INTEGRAL AMPULE

(71) Applicant: TELEFLEX MEDICAL INCORPORATED, Research Triangle Park, NC (US)

(72) Inventors: Chris KORKUCH, Chester Springs, PA (US); Jeffrey P. KUEHN, Schuylkill Haven, PA (US)

(21) Appl. No.: 14/474,605

(22) Filed: Sep. 2, 2014

Related U.S. Application Data
(60) Provisional application No. 61/872,027, filed on Aug. 30, 2013.

(51) Int. Cl. A61M 5/24; A61M 5/31; A61M 5/315

(52) U.S. Cl. CPC A61M 5/2466; A61M 5/31581; A61M 5/3145; A61M 5/31511; A61M 2005/2474; A61M 2005/3128
USPC 604/506; 604/193; 604/190

(57) ABSTRACT

A syringe having an integral ampule is disclosed. The syringe includes a barrel defining a reservoir configured to store a fluid and a plunger defining an inner cavity. The plunger includes an ampule within the inner cavity, where the ampule seals the fluid. The plunger also includes a cap at a proximal end of the plunger that is configured to move distally relative to the plunger, and a first one-way valve at the distal end of the plunger. The distal movement of the cap relative to the plunger causes an opening within the ampule. A method of injecting a fluid within a patient using a syringe having an integral ampule is also disclosed.

### CROSS REFERENCE TO RELATED APPLICATION

This application claims the benefit of U.S. Provisional Application No. 61/872,027, filed on Aug. 30, 2013, the disclosure of which is hereby incorporated by reference, in its entireties.

### TECHNICAL FIELD

This disclosure generally relates to medical devices for administering a fluid, such as a liquid medicament, in a patient. More particularly, this disclosure relates to a syringe having an integral ampule storing a fluid to be administered to a patient, resulting in increased procedural efficiency and safety, as well as decreased risk of contamination, relative to known syringes.

### BACKGROUND

Liquid medicaments, such as saline, drugs, and anesthetics, are typically stored in sealed ampules before administration to prevent contamination of the liquid medicaments. To administer a liquid medicament, a clinician first cracks the top of the sealed ampule, withdraws the liquid medicament from the ampule into the barrel of a syringe, inserts the needle of the syringe into a patient, and depresses the plunger of the syringe to inject the liquid medicament into the patient. Such a procedure is time consuming and poses several risks to the clinician and the patient.

In particular, with regard to clinician and patient safety, because many liquid medicaments are reactive, the ampules are typically made of an inert material, such as glass, to increase the shelf life of the liquid medicaments. By cracking a glass ampule, however, small glass shards from the broken ampule can contaminate the liquid medicament, which can be harmful to the patient. In addition, the cracked region of the glass ampule presents risks of sharps injuries to the clinician before disposal of the broken ampule. Moreover, once the ampules are cracked, bacteria can contaminate the liquid medicament through the opening formed in the ampule, which is especially risky in healthcare settings where the atmospheric air is contaminated with harmful bacteria and viruses. In addition, injection kits include several different parts, such as an ampule cracker and a filter straw, in addition to the syringe and ampule. The multiple separate parts increase the risk of contamination.

Further, with regard to procedural efficiency, in addition to the time required to crack the ampule, the clinician must also remove air and/or air bubbles within the barrel of the syringe prior to injection, which increases the amount of clinician time required per injection. The clinician must also dispose of the different parts of the injection kit in different containers. For example the cracked ampule and syringe needle must be disposed in sharps disposal containers.

Therefore, a need exists for a syringe having an integral ampule that reduces the risk of contamination and injury to the clinician and patient, while increasing the procedural efficiency of injection.

### SUMMARY

The foregoing needs are met, to a great extent, by implementations of the syringe having an integral ampule according to this disclosure. In accordance with one implementation, a syringe includes a barrel defining a reservoir configured to store a fluid and a plunger defining an inner cavity. The plunger includes an ampule within the inner cavity, where the ampule seals the fluid. The plunger also includes a cap at a proximal end of the plunger that is configured to move distally relative to the plunger, and a first one-way valve at the distal end of the plunger. The distal movement of the cap relative to the plunger causes an opening within the ampule.

In some implementations, the distal end of the plunger can include a filter that is proximal of the first one way valve. Rotation of the cap can cause the distal movement of the cap relative to the plunger. The distal end of the cap can include external threads that are received within internal threads at the proximal end of the plunger.

In some implementations, the syringe can further include a needle hub configured to be connected to a distal end of the barrel of the syringe, and a needle extending distally from the needle hub. The needle hub can also further include a second one-way filter configured to permit fluid flow only in the distal direction and a needle safety housing that is configured to move distally relative to the needle to cover a sharp distal tip of the needle.

In some implementations, the first one-way valve can be configured to permit fluid flow only in the distal direction. A predetermined region at a distal end of the ampule can be configured to open in response to the distal movement of the cap relative to the plunger. The wall thickness of a distal end of the ampule can be less than the wall thickness of a proximal end of the ampule. The inner cavity of the plunger can include a pointed structure configured to break the ampule in response to the distal movement of the cap relative to the plunger.

In some implementations, the ampule can be made of a flexible material, and the flexible ampule can be configured to sever in response to the distal movement of the cap relative to the plunger. The flexible ampule can be configured to sever in response to the distal movement of the cap relative to the plunger by contacting a sharp structure within the inner cavity of the plunger in response to the distal movement of the cap relative to the plunger.

In some implementations, at least a portion of the walls of the ampule can be scored. The cap can further define an air vent. The air vent can be a longitudinal bore running along the entire longitudinal length of the cap. The cap can be configured to be removably connected to the plunger.

According to another implementation, a method of using a syringe having an integral ampule is disclosed. Initially, a clinician receives the syringe having an integral ampule. The syringe includes a barrel and a plunger. The barrel defines a reservoir, and the plunger includes an ampule sealing a fluid and a cap. Distal movement of the cap relative to the plunger is caused, where the distal movement of the cap causing an opening within the ampule. Next, the plunger is caused to move in a proximal direction to cause the fluid to pass through the plunger into the reservoir defined by the barrel. Finally, the plunger is caused to move in a distal direction to cause the fluid in the reservoir to pass through the syringe.

In some implementations, the received syringe can be a prefilled syringe including the ampule sealing the fluid within the plunger. In other implementations, the received syringe does not include the ampule and the clinician initially can remove the cap from the plunger, can insert the ampule storing the fluid within the plunger, and can apply the cap to the plunger.

In some implementations, the distal movement of the cap relative to the plunger can be caused by rotating the cap of the plunger. The plunger can be caused to move in the proximal direction by pulling the plunger in the proximal direction. The plunger can be caused to move in the distal direction by pushing the plunger in the distal direction.

Certain implementations of the syringe having the integral ampule have been outlined so that the detailed description below may be better understood. There are, of course, additional implementations that will be described below and which will form the subject matter of the claims.

In this respect, before explaining at least one implementation in detail, it is to be understood that the syringe having the integral ampule is not limited in its application to the details of construction and to the arrangements of the components set forth in the following disclosure or illustrated in the drawings. Also, it is to be understood that the phraseology and terminology employed herein, as well as in the Abstract, are for the purpose of description and should not be regarded as limiting.

As such, those skilled in the art will appreciate that the conception upon which this disclosure is based may readily be utilized as a basis for the designing of other structures, methods, and systems for carrying out the several purposes of the syringe having the integral ampule. It is understood, therefore, that the claims include such equivalent constructions insofar as they do not depart from the spirit and scope of the present disclosure.

### BRIEF DESCRIPTION OF THE DRAWINGS

FIG. 1a illustrates a cross-sectional view of an implementation of a syringe having an integral ampule taken along its longitudinal axis.

FIG. 1b illustrates a cross-sectional view of an implementation of a needle hub connected to the syringe having the integral ampule taken along the longitudinal axis of the syringe.

Implementations of the syringe having the integral ampule are described with reference to the drawings, in which like reference numerals refer to like parts throughout.

### DETAILED DESCRIPTION

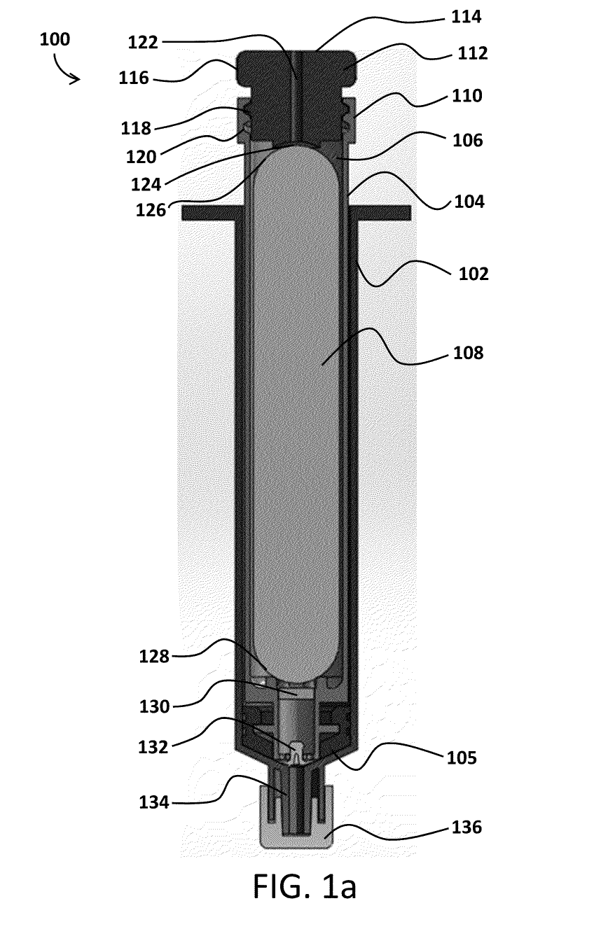

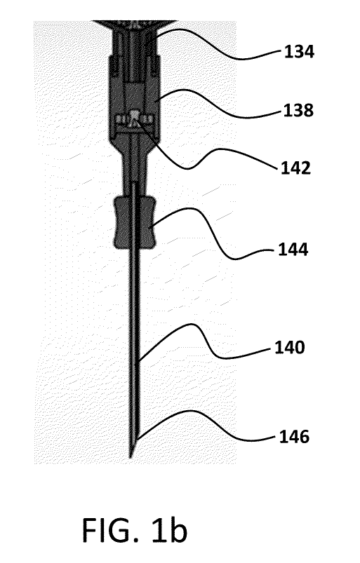

Referring to FIG. 1a, a cross-sectional view of an implementation of a syringe 100 having an integral ampule taken along its longitudinal axis is illustrated. The syringe 100 includes a barrel 102 and a plunger 104 that is received within the barrel 102. The barrel 102 and plunger 104 of the syringe 100 can be made of plastic, such as a polymer, or glass. The plunger 104 includes a distal seal 105 that is in contact with the inner surface of the barrel 102 to seal the reservoir defined by the barrel 102 and the distal seal 105. The plunger 104 defines an inner cavity 106 that receives an ampule 108 storing a fluid. The fluid can be a liquid, a gas, a gel, a slurry, an emulsion, or a suspension and is, preferably, a liquid medicament. The ampule 108 can be made of any material that can be broken or severed and, preferably, can be made of glass. The proximal end 110 of the plunger 104 includes a cap 112 that can be twisted by the clinician to break or sever the ampule 108.

In some implementations, the ampule 108 can be placed within the inner cavity 106 of the plunger 104 before the cap 112 is applied to seal the inner cavity 106, i.e., before packaging of the syringe 100. Such a syringe is commonly referred to as a prefilled syringe. In other implementations, the syringe 100 can be packaged without the ampule 108. A clinician can then remove the syringe 100 from its packaging, then unscrew the cap 112, insert the ampule 108 with the fluid of choice, and then screw the cap 112 back on the proximal end 120 of the plunger 104.

The cap 112 includes a flat proximal surface 114 to which a clinician can apply a distal force to move the plunger 104 in the distal direction. A distal force applied to the flat proximal surface 114 by the clinician will cause distal movement of the plunger 104, but not distal movement of the cap 112 relative to the plunger 104 due to the threaded attached attachment of the cap 112 to the plunger 104. In some implementations, as shown in FIG. 1, the cap 112 can have a circular cross-section in a plane perpendicular to its longitudinal axis. In other implementations, the cross-section of the cap 112 can have an oval, a square, or other geometric shape in the plane perpendicular to its longitudinal axis.

In some implementations, the outer wall of the proximal portion 116 of the cap 112 can include gripping features to improve grip of the cap 112 while the clinician is twisting the cap 112. The gripping features can be, for example, depressions, projections, a textured surface, and/or a different material, such as rubber, that covers the outer wall of the proximal portion 116 of the cap 112. The distal region 118 of the cap 112 includes external threads along its outer wall that are received within corresponding internal threads along the proximal end 120 of the plunger 104. The cap 112 also includes an air vent 122 that allows air to pass through the cap 112 into the inner cavity 106 defined by the plunger 104. In some implementations, as illustrated in FIG. 1, the air vent 122 can be a central longitudinal bore running along the entire longitudinal length of the cap 112. In other implementations, the cap 112 can include multiple longitudinal bores located adjacent the circumference of the cap 112. In some implementations, the air vent 122 can include a one-way valve (not shown) to only allow atmospheric air to enter the inner cavity 106 while not allowing fluid to exit the inner cavity 106. The one-way valve of the air vent 122 can be any type of one-way valve, including a flap valve, a ball valve, a duck-bill valve, a slit valve, an umbrella valve, etc.

In some implementations, the distal surface 124 of the cap 112 can be shaped to complement the proximal surface 126 of the ampule 108. For example, as shown in FIG. 1, the distal surface 124 of the cap 112 is curved to maximize the area of the distal surface 124 contacting the curved proximal surface 126 of the ampule 108. In other implementations where the proximal surface 126 of the ampule 108 is flat, the distal surface 124 of the cap 112 can also be flat. As a result of the complementary shape of the distal surface 124, the longitudinal force applied by the cap 112 can be evenly spread to the ampule 108 over the contact area between the cap 112 and the ampule 108.

Similarly, as shown in FIG. 1, the distal end 128 of the ampule 108 is shown to be curved, but can, in some implementations, be flat. In addition, is some implementations, the ampule 108 can have a neck at its proximal end and be placed upside-down in the plunger 104, so that the neck is distal of the base of the ampule 108. In such implementations, the ampule 108 can be configured to break at its neck by an angled platform located at the distal end of the inner cavity 106. As such, the distal force applied by the cap 112 will force the neck of the ampule 108 against the angled platform, causing the neck to break.

The cap 112 is configured to be rotated by the clinician to impart distal movement of the cap 112 relative to the plunger 104. The distal movement of the cap 112 applies a longitudinal force to the ampule 108, causing the ampule 108 to break or sever when the longitudinal force applied by the cap 112 is greater than the force required to break a predetermined region of the ampule 108. The opening created within the ampule 108 in turn releases the fluid stored in the ampule 108 so that it can be received within the reservoir defined by the barrel 102.

In some implementations, predetermined regions of the walls of the ampule 108 can be thinner than other regions of the walls of the ampule 108 to control breakage of the ampule 108 in the predetermined regions. For example, preferably, the walls of the distal end 128 of the ampule 108 can be thinner than the side and proximal walls of the ampule 108, such that the distal end 128 of the ampule 108 breaks before the other regions of the ampule 108 under the longitudinal force applied by the cap 112. In some implementations, all or part of the circumference at a longitudinal region of the ampule 108 can be scored to create a weakened or frangible region of the ampule 108 configured to break more quickly upon force exerted from the cap 112. For example, a circumferential score line can be formed at the distal end 128 of the ampule 108.

In other implementations, the walls of the ampule 108 can have a consistent thickness and a pointed or sharp structure (not shown) can be included in the inner cavity 106. Therefore, under the longitudinal force from the cap 112, the force concentrated against the pointed or sharp structure will cause breakage of the ampule at the region aligned with the pointed or sharp structure.

Following breakage of the ampule 108 and release of the fluid within the ampule 108, the clinician can pull proximally on the plunger 104 or cap 112 to permit the fluid to flow from the inner cavity 106 into the reservoir defined by the barrel. In particular, a vacuum is created within the distal end of the barrel 102 as the clinician pulls proximally on the plunger 104. The vacuum draws the fluid through the filter 130 and the first one-way valve 132 into the barrel 102. As the fluid is drained, air is sucked from the atmosphere through the air vent 122 to replace the volume of the fluid exiting the inner cavity 106.

The filter 130 prevents fragments of the broken ampule 108 to pass from the inner cavity 106 into the barrel 102. The filter 130 can also filter other impurities that may be present in the fluid, depending on the pore size of the filter 130. In some implementations, the filter 130 can be a single layer filter, while in other implementations, the filter 130 can include multiple stacked layers.

The first one-way valve 132 can be any type of one-way valve, including a flap valve, a ball valve, a duck-bill valve, a slit valve, an umbrella valve, etc. The first one-way valve 132 can be configured to be biased closed when a vacuum is not formed within the reservoir of the barrel 102 and to open when the vacuum is formed.

The distal end of the barrel 102 may include a Luer-lock nozzle 134 that is sealed by a protective cap 136. In some implementations, the syringe 100 can be packaged with the protective cap 136 and the clinician can remove the protective cap 136 and attach needle hub 138 to the Luer-lock nozzle 134 to prepare the syringe 100 for injection. In other implementations, the syringe 100 may not include the protective cap 136 and the needle hub 138 may be connected to the Luer-lock nozzle 134 at the factory. In such implementations, the needle 140 extending distally from the needle hub 138 can be covered by a needle guard (not shown). In some implementations, the syringe 100 may not include the Luer-lock nozzle 134 and the needle hub 138 may be integral with the barrel 102.

In particular, FIG. 1b illustrates a cross-sectional view of an implementation of the needle hub 138 connected to the Luer-lock nozzle 134 taken along the longitudinal axis of the syringe 100. The needle hub 138 includes a second one-way valve 142 that allows the fluid in the barrel 102 to pass through the needle hub 138 to the needle 140 for injection in the patient when the clinician presses down on the plunger 104 or the cap 112. The second one-way valve 142 prevents fluids from the patient, such as blood, to enter the barrel 102 of the syringe 100 and contaminate the fluid. The second one-way valve 142 can be any type of one-way valve, including a flap valve, ball valve, a duck-bill valve, a slit valve, an umbrella valve, etc. In some implementations, the second one-way valve 142 can be the same type as the first one-way valve 132, while in other implementations, the second one-way valve 142 can be a different type. The second one-way valve 142 can be configured to be biased closed when a positive pressure is not applied to the fluid within the barrel 102 and to open when positive pressure is applied to the fluid within the barrel 102 by the clinician.

The needle hub 138 also includes a needle safety housing 144. The needle safety housing 144 can be moved distally relative to the needle 140 following injection by the clinician to prevent needle pricks. The needle safety housing 144 circumferentially covers the sharp distal tip 146 of the needle 140 when in its distal position.

Following attachment of the needle hub 138, the clinician presses down on the plunger 104 or cap 112 to force the fluid within the plunger 104 out of the sharp distal tip 146 of the needle 140. Following injection of the desired amount of the fluid, the clinician can move the needle safety housing 144 over the sharp distal tip 146 of the needle 140 and dispose of the syringe 100.

The many features and advantages of the syringe 100 are apparent from the detailed specification, and thus, the claims cover all such features and advantages within the scope of this application. Further, numerous modifications and variations are possible. For example, although two one-way valves are illustrated in FIGS. 1a-b, the syringe 100 can include only the first one-way valve 132 or three or more one-way valves.

In another example, the cap 112 can be moved distally relative to the plunger 104 by longitudinal force applied by the clinician to the cap as opposed to rotation the cap 112. In such an example, the cap 112 would not be connected to the plunger 104 by a threaded connection, but can be connected by a friction fit, for example.

In yet another example, the syringe 100 may not include a cap 112 configured to apply a longitudinal force to crack the ampule 108. Instead, the syringe 100 may include a contact member, such as a rod, extending from the side walls of the plunger 104 and configured to apply a perpendicular force to the side walls of the ampule 108 to break the ampule 108. The contact member can be forced into the side walls of the ampule 108 by direct force from the clinician or by actuation of a lever, for example.

In still another example, the plunger 104 can be made of a flexible material such that flexing of the plunger by the clinician can break the ampule 108. In such an example, the plunger 104 can initially be separate from the barrel 102. The clinician can then flex the plunger 104 to break the ampule and then insert the plunger 104 within the barrel 102. In this example, the fluid with the inner cavity 106 would not escape from the plunger 104 due to the first one-way valve 132 at the proximal end of the plunger 104.

Although the ampule 108 has been described as being broken upon the excursion of force, in some implementations, the ampule 108 can be flexible. The ampule 108 can, for example, be made of a flexible plastic. In such examples, the plunger 104 and/or cap 112 can be configured to puncture the ampule 108 to release the fluid from the ampule 108 to the inner cavity 106. For example, the distal portion of the inner cavity 106 can include a sharp object that can puncture the distal end 128 of the ampule 108. In another example, the sharp object can be connected to the cap 112, such that distal movement of the cap 112 will result in puncturing of the proximal surface 126 of the ampule 108.

Although a single ampule 108 has been described, two or more ampules can be held within the plunger 104. For example, a first ampule can be filled with a granulated or powdered medicament and a second ampule can be filled with a liquid, such as saline. In such an example, both ampules can be broken simultaneously or sequentially to mix the contents of both ampules. In one example, the distal movement of the cap 112 can cause the first, more proximal ampule to break and then the second, more distal ampule to break. In another example, two ampules can be placed in parallel along their longitudinal axes. The distal movement of the cap 112 can then cause both ampules to break simultaneously. When the clinician pulls proximally on the plunger 104 or cap 112 to permit the fluid within the inner cavity 106 to flow into the reservoir defined by the barrel, the solid medicament is mixed with the liquid to form a reconstituted homogenous solution.

In another example, the two or more ampules can contain separate liquids to be mixed prior to injection. When mixed, the combination of the liquids can have limited efficacy life and, therefore, they can be kept in separate ampules within the plunger 104. Following breakage of the ampules, when the clinician pulls proximally on the plunger 104 or cap 112 to permit the fluid within the inner cavity 106 to flow into the reservoir defined by the barrel, the separate liquids are mixed.

As such, it is not desired to limit the syringe 100 to the exact construction and operation described and illustrated and, accordingly, all suitable modifications and equivalents may fall within the scope of the claims.

What is claimed is:

1. A syringe having an integral ampule, the syringe comprising:
a barrel defining a reservoir configured to store a fluid; and
a plunger defining an inner cavity and configured to be received within the barrel, the plunger comprising:
an ampule within the inner cavity, the ampule sealing the fluid,
a cap at a proximal end of the plunger and being configured to move distally relative to the plunger, and
a first one-way valve at a distal end of the plunger,
wherein distal movement of the cap relative to the plunger causes an opening within the ampule.

2. The syringe of claim 1, wherein the distal end of the plunger further comprises a filter that is proximal of the first one way valve.

3. The syringe of claim 1, wherein rotation of the cap causes the distal movement of the cap relative to the plunger.

4. The syringe of claim 3, wherein a distal end of the cap comprises external threads that are received within internal threads at the proximal end of the plunger.

5. The syringe of claim 1, further comprising:
a needle hub configured to be connected to a distal end of the barrel of the syringe; and
a needle extending distally from the needle hub.

6. The syringe of claim 5, wherein the needle hub further comprises a second one-way filter configured to permit fluid flow only in a distal direction.

7. The syringe of claim 5, wherein the needle hub further comprises a needle safety housing that is configured to move distally relative to the needle to cover a sharp distal tip of the needle.

8. The syringe of claim 1, wherein the first one-way valve is configured to permit fluid flow only in a distal direction.

9. The syringe of claim 1, wherein a predetermined region at a distal end of the ampule is configured to open in response to the distal movement of the cap relative to the plunger.

10. The syringe of claim 1, wherein the wall thickness of a distal end of the ampule is less than the wall thickness of a proximal end of the ampule.

11. The syringe of claim 1, wherein the inner cavity of the plunger comprises a pointed structure configured to break the ampule in response to the distal movement of the cap relative to the plunger.

12. The syringe of claim 1, wherein the ampule is made of a flexible material, and the flexible ampule is configured to sever in response to the distal movement of the cap relative to the plunger.

13. The syringe of claim 12, wherein the flexible ampule is configured to sever in response to the distal movement of the cap relative to the plunger by contacting a sharp structure within the inner cavity of the plunger in response to the distal movement of the cap relative to the plunger.

14. The syringe of claim 1, wherein at least a portion of the walls of the ampule is scored.

15. The syringe of claim 1, wherein the cap further defines an air vent.

16. The syringe of claim 15, wherein the air vent is a longitudinal bore running along the entire longitudinal length of the cap.

17. The syringe of claim 1, wherein the cap is configured to be removably connected to the plunger.

18. A method for injecting a fluid within a patient using a syringe having an integral ampule, the method comprising:
receiving a syringe comprising a barrel and a plunger, the barrel defining a reservoir, and the plunger comprising an ampule sealing a fluid and a cap;
causing distal movement of the cap relative to the plunger, the distal movement of the cap causing an opening within the ampule;
causing the plunger to move in a proximal direction to cause the fluid to pass through the plunger into the reservoir defined by the barrel; and
causing the plunger to move in a distal direction to cause the fluid in the reservoir to pass through the syringe.

19. The method of claim 18, wherein receiving the syringe comprises receiving a prefilled syringe comprising the ampule sealing the fluid within the plunger.

20. The method of claim 18, wherein receiving the syringe comprises:
removing the cap from the plunger;
inserting the ampule storing the fluid within the plunger; and
applying the cap to the plunger.

21. The method of claim 18, wherein the syringe further comprises a needle connected to a distal end of the barrel.

22. The method of claim 18, wherein causing the distal movement of the cap relative to the plunger comprises rotating the cap of the plunger to cause the distal movement of the cap relative to the plunger.

23. The method of claim 18, wherein causing the plunger to move in the proximal direction comprises pulling the plunger in the proximal direction.

24. The method of claim 18, wherein causing the plunger to move in the distal direction comprises pushing the plunger in the distal direction.
